(19) United States
(12) Patent Application Publication
(10) Pub. No.: US 2012/0142676 A1
(43) Pub. Date: Jun. 7, 2012

(54) Oxathiazine and dithiine oxides as inhibitors of sulfhydryl-dependent biomolecules

(76) Inventors: Gaik-Lean Chee, Winnipeg (CA); Walter G. Brouwer, Guelph (CA); Ewa Osika, Cambridge (CA); Brian B. Hasinoff, Winnipeg (CA); A. David Brewer, Puslinch (CA)

(21) Appl. No.: 13/136,264

(22) Filed: Jul. 27, 2011

Related U.S. Application Data
(60) Provisional application No. 61/374,672, filed on Aug. 18, 2010.

(51) Int. Cl. A61K 31/54; C07D 419/04; A61P 35/00; A61K 31/541; A61K 31/385; C07D 291/06; C07D 339/08

(52) U.S. Cl. 514/222.5; 544/2; 549/21; 514/436

(57) ABSTRACT

Novel derivatives of dihydro-1,4,2-oxathiazine and dihydro-1,4-dithiine oxides, more particularly, novel derivatives of dihydro-1,4,2-oxathiazine and dihydro-1,4-dithiine oxides that target cysteine residues of biomolecules of pharmacological importance are provided as pharmaceutically useful compounds, for example, as anticancer, antiinfectious, antigastric acid secretion, antiosteoporosic, and antiinflammatory agents.

### BACKGROUND OF THE INVENTION

1. Field of the Invention

The present invention relates to new derivatives of dihydro-1,4,2-oxathiazine and dihydro-1,4-dithiin oxides. More particularly, the invention relates to derivatives of dihydro-1,4,2-oxathiazine and dihydro-1,4-dithiine oxides that target cysteine residues of biomolecules of pharmacological importance. Therefore, these derivatives may be pharmaceutically useful as anticancer, antiinfectious, antigastric acid secretion, antiosteoporosic, and antiinflammatory agents.

2. Description of Related Art

Biomolecules containing the cysteine residues critical for their normal biological functions are important targets for various classes of chemotherapeutic agents (reviewed by Leung-Toung, R., Li, W., Tam, T. F., Karimian, K. “Thiol-dependent enzymes and their inhibitors: A review”. Current Medicinal Chemistry (2002), 9, 979-1002; Scozzafava, A., Mastrolorenzo, A., Supuran, C. T. “Agents that target cysteine residues of biomolecules and their therapeutic potential”. Expert Opinion on Therapeutic Patents (2001), 11, 765-787; and Scozzafava A.; Casini A.; Supuran C. T. “Targeting cysteine residues of biomolecules: New approaches for the design of antiviral and anticancer drugs”. Current Medicinal Chemistry (2002), 9, 1167-1185). Some examples of these types of biomolecules are DNA topoisomerases, DNA and RNA polymerases, cysteine proteases, alcohol dehydrogenase, carbonic anhydrase, H⁺/K⁺ ATPase, and certain kinases. These enzymes are involved in many different disease processes such as cancer cell proliferation, microbial infection, excess acid secretion, bone loss and inflammation. The sulfhydryl groups of these biomolecules may participate in oxidative-reductive processes that lead to biomolecular conformational changes of pharmacological consequences. The sulfhydryl groups may also form organometallic bonds with Zn(II), Cu(II), and Fe(III) important for enzymatic catalysis as in the case of metallo-enzymes. The sulfhydryl groups may also act as nucleophiles in promoting peptide bond cleavage as exemplified by cysteine proteases. Therefore, drugs targeting the cysteine residues of biomolecules have applications as therapeutic agents for treating disease disorders resulted from the actions of these biomolecules.

Dihydro-1,4,2-oxathiazine and dihydro-1,4-dithiin oxides in the present invention exhibited reactivity as electrophiles toward biomolecules containing sulfhydryl groups at physiological conditions to form covalent adducts. The sulfhydryl-targeting ability of these compounds strongly implicates therapeutic effects in treating disease disorders mediated by the biomolecules containing critical sulfhydryl groups. Their practical use as therapeutic agents has been demonstrated by their potent cytotoxicity toward human leukemia K562 cells and inhibition of DNA topoisomerase II enzyme catalytic activity. These activities confirm the compounds as effective anticancer agents.

Some dihydro-1,4,2-oxathiazine and dihydro-1,4-dithiine oxides have been disclosed for use as herbicides, biocides, plant desiccants, and defoliants in agricultural and industrial biocidal applications (U.S. Pat. Nos. 4,569,690; 5,777,110; 5,712,275; 3,920,438; 3,997,323; 4,004,018; and 4,097,580). A group of dithiine tetraoxides was disclosed as galanin receptor antagonists for treating disorders of the central nervous system (U.S. Pat. No. 6,407,136), and as inhibitors of gastric acid secretion (U.S. Pat. No. 4,109,006). A dihydro-1,4,2-oxathiazine oxide, bethoxazin, was disclosed in a cytotoxic composition comprising an actophosphatase inhibitor for increasing cellular uptake of biocidal bethoxazin (PCT WO 2005/014777). However, oxathiazine and dithiine oxides have not been disclosed as inhibitors of sulfhydryl-dependent biomolecules. Moreover oxathiazine and dithiine oxides in the present invention have not been disclosed as useful for human pharmaceutical applications, in particular but not limited to anticancer, antiinfectious, antigastric acid secretion, antiosteoporosic, and antiinflammation applications.

### SUMMARY OF THE INVENTION

This invention relates to a compound of the formula:

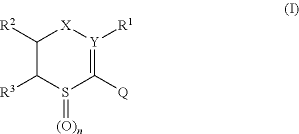

Wherein X is oxygen or sulfone; Y is nitrogen when X is oxygen, or carbon when X is sulfone; n is 1 or 2, with the proviso that when n is 1, X must be oxygen and Y must be nitrogen; R¹ is present when Y is carbon, or absent when Y is nitrogen, and when present is hydrogen, C₁-C₆ linear or branched alkyl, phenyl, trihalomethyl, cyano, benzyl, phenylsulfone, methyl sulfone, methyl alcohol, nitro, methylene C₁-C₆ alkoxy, methylene C₁-C₆ thioalkoxy, methylene benzyloxy, methylene phenoxy, or methylene acetate; R² and R³ are each independently hydrogen, C₁-C₆ linear or branched alkyl, benzyl, methylene C₁-C₄ alkoxy, or halogen; Q is (a) phenyl, with the proviso that R¹ is present and cannot be hydrogen, straight chain or branched alkyl, or substituted or unsubstituted phenyl;

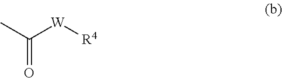

wherein W is oxygen, amino, C₁-C₅ linear or branched alkylamino, or CH₂; R⁴ is hydrogen, C₁-C₆ linear or branched alkyl, C₅-C₆ cycloalkyl, ethylphenyl, or phenyl optionally substituted with 1 to 3 substituents independently selected from halogen, C₁-C₆ alkyl, C₁-C₆ alkoxyl, C₁-C₆ linear or branched alkoxylcarbonyl, trihalomethyl, phenyl, nitro, or aminoacetyl, with the proviso that R¹ may be present and if present cannot be hydrogen, straight chain or branched alkyl, cyano, or substituted or unsubstituted phenyl;
(c) 1,3,4-oxadiazole substituted with phenyl or halophenyl;

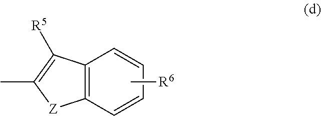

wherein Z is sulfur, sulfoxide, or sulfone; R⁵ is hydrogen, C₁-C₄ linear alkyl, C₁-C₄ branched alkoxy, halogen, haloalkyl, nitro, phenyl, or methylene halophenoxy; R⁶ is hydrogen, C₁-C₄ alkyl, halogen, or trihalomethyl, with the proviso that when Z is sulfur, R⁵ cannot be hydrogen or C₁-C₄ branched alkoxy, and R⁶ cannot be hydrogen or C₁-C₄ alkyl;

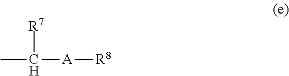

wherein A is oxygen or sulfone; R⁷ is hydrogen or C₁-C₄ alkoxy; R⁸ is C₁-C₆ linear or branched alkyl, phenyl, biphenyl, halophenyl, C₁-C₄ alkylphenyl, benzyl, C₁-C₄ alkylcarbonyl, phenylcarbonyl, C₁-C₄ alkylaminocarbonyl, or phenylaminocarbonyl.

The present invention also relates to a method of treating a human disease disorder mediated by a sulfhydryl-dependent biomolecule in a subject in need thereof comprising administering to the subject an effective amount of a compound of structural formula:

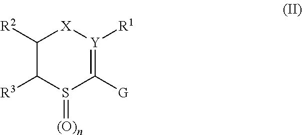

Wherein X is oxygen or sulfone; Y is nitrogen when X is oxygen, or carbon when X is sulfone; n is 1 or 2, with the proviso that when n is 1, X must be oxygen and Y must be nitrogen; R¹ is present when Y is carbon, or absent when Y is nitrogen, and when present is a hydrogen, C₁-C₆ linear or branched alkyl, C₁-C₃ haloalkyl, trihalomethyl, benzyl, phenylsulfone, methyl sulfone, methyl alcohol, nitro, methylene C₁-C₆ alkoxy, methylene C₁-C₆ thioalkoxy, methylene benzyloxy, methylene phenoxy, methylene acetate, C₁-C₆ alkoxycarbonyl, phenyl, nitrophenyl, halophenyl, C₁-C₄ alkylphenyl, C₁-C₄ alkoxyphenyl, or naphthyl; R² and R³ are each independently hydrogen, C₁-C₆ linear or branched alkyl, benzyl, methylene C₁-C₄ alkoxy, or halogen; G is
(a)
phenyl, naphthyl or pyridinyl; phenyl optionally substituted with 1 to 3 substituents independently selected from halogen, C₁-C₁₂ linear or branched alkyl, C₅-C₆ cycloalkyl, haloalkyl, phenyl, C₁-C₄ alkoxy, C₁-C₄ thioalkoxy, tetrahydrophyranyloxy, phenoxy, C₁-C₅ alkylcarbonyl, C₁-C₅ alkoxycarbonyl; C₁-C₅ alkylaminocarbonyl, phenylaminocarbonyl, tolylamonicarbonyl, morpholinocarbonyl, amino, nitro, cyano, dioxolanyl;

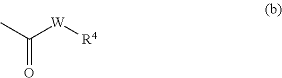

wherein W is oxygen, amino, C₁-C₅ linear or branched alkylamino, or CH₂; R⁴ is hydrogen, C₁-C₆ linear or branched alkyl, C₅-C₆ cycloalkyl, ethylphenyl, or phenyl optionally substituted with 1 to 3 substituents independently selected from halogen, C₁-C₆ alkyl, C₁-C₆ alkoxyl, C₁-C₆ linear or branched alkoxylcarbonyl, trihalomethyl, phenyl, nitro, or aminoacetyl, with the proviso that R¹ cannot be hydrogen, straight chain or branched alkyl, or cyano;
(c) thienyl or furanyl, each optionally substituted with 1 to 3 substituents independently selected from halogen, C₁-C₆ linear or branched alkyl, C₁-C₅ alkoxy, C₁-C₅ thioalkoxy, trihalomethyl, C₁-C₆ alkoxycarbonyl, cyano, acetyl, formyl, benzoyl, nitro, phenylaminocarbonyl, or phenyl;
(e) 1,3,4-oxadiazole substituted with phenyl or halophenyl;

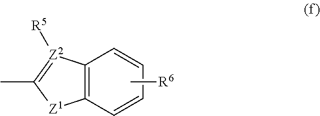

wherein Z¹ is oxygen, sulfur, sulfoxide, or sulfone; Z² is nitrogen or carbon; R⁵ is present when Z² is carbon, or absent when Z² is nitrogen, and when present is a hydrogen, C₁-C₄ linear alkyl, C₁-C₄ branched alkoxy, halogen, haloalkyl, nitro, phenyl, or methylene halophenoxy; R⁶ is hydrogen, C₁-C₄ alkyl, halogen, or trihalomethyl;

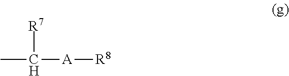

wherein A is oxygen or sulfone; R⁷ is hydrogen or C₁-C₄ alkoxy; R⁸ is C₁-C₆ linear or branched alkyl, phenyl, biphenyl, halophenyl, C₁-C₄ alkylphenyl, benzyl, C₁-C₄ alkylcarbonyl, phenylcarbonyl, C₁-C₄ alkylaminocarbonyl, or phenylaminocarbonyl.

### BRIEF DESCRIPTION OF THE DRAWINGS

FIG. 1 depicts the experimental and calculated molecular weights of peaks observed in the deconvoluted molecular ion regions of HSA treated with DMSO and Compound 9, as shown in spectra B and D, respectively. Panels A and C are ESI positive-ion mass spectra of HSA and HSA treated with compound 9, respectively.

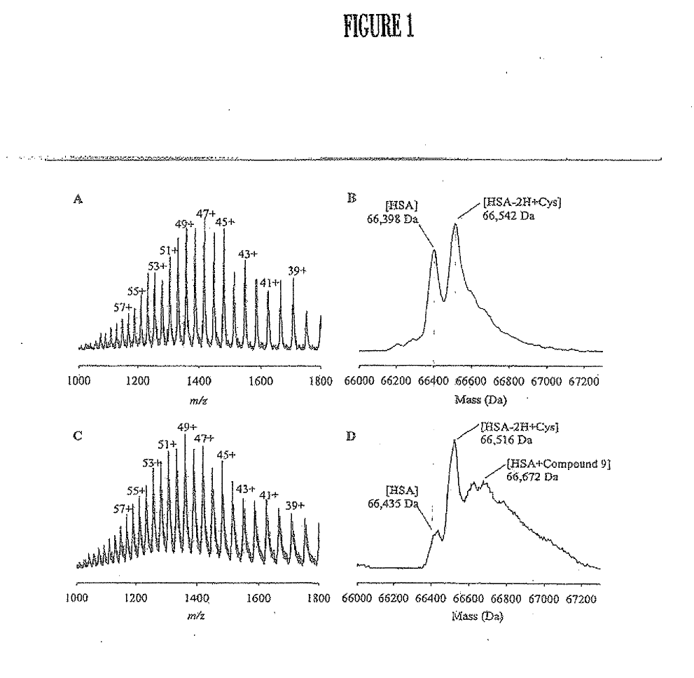

### DETAILED DESCRIPTION OF THE INVENTION

The preparation of compounds of formula I and II can be achieved using procedures analogous to those described in U.S. Pat. No. 4,569,690; 5,777,110; 4,004,018; 4,097,580; and 3,997,323, the disclosures of which are incorporated herein by reference, or the procedures described in Examples 1-9 below. The chemical reactivity of compounds of formula I and II toward naturally occurring sulfhydryl compounds such as cysteine and glutathione, and cysteine-containing biomolecules can be studied using Examples 10-11 below. The biological activities of compounds of formula I and II in cancer cells and against an essential human enzyme containing critical sulfhydryl groups can be studied using Examples 12-13 below.

Compounds of formula I and II in the present invention exhibited reactivity as electrophiles toward synthetic cysteine and glutathione, human serum albumin protein and cellular proteins containing sulfhydryl groups at physiological conditions, blocking the ability of these biomolecules from reacting with a fluorescent probe reactive toward sulfhydryl compounds, as exemplified in Table 4. Compounds of formula I and II in the present invention react with sulfhydryl biomolecules such as human serum albumin protein to form covalent adducts, as exemplified in Table 5. Therefore, these compounds are regarded as sulfhydryl-targeting compounds. Their pharmaceutical applications as therapeutic agents have been demonstrated by their potent cytotoxicity toward human leukemia K562 cells and inhibition of DNA topoisomerase II enzyme catalytic activity in the low micromolar concentration range, as exemplified in Table 6.

### EXAMPLES

### Examples of Compound Synthesis

### Example 1

### Preparation of 4-oxo-5,6-dihydro-N-phenyl-1,4λ⁴,2-oxathiazine-3-carboxamide (Compound #3)

### Step 1: Preparation of 2-(methylsulfanyl)-N-phenyl-2-sulfanylideneacetamide

2-Chloro-N-phenylacetamide (10 g) in DMF (13 ml) was added dropwise to a stirred mixture of sulfur (4 g), DMF (20 ml) and triethylamine (25 ml). The resulting red solution was stirred at room temperature overnight, concentrated under vacuum to remove excess triethylamine, added dropwise methyl iodide (4 ml), and stirred again at room temperature overnight before it was poured on ice. The resulting red solid was filtered, washed with water, dried, and further washed with diethyl ether (8 g, mp 77-79° C.).

### Step 2: Preparation of N-phenyl-5,6-dihydro-1,4,2-oxathiazine-3-carboxamide

A solution of triethylamine (3 ml)/methanol (9 ml) was added dropwise to a stirred suspension of 2-(methylsulfanyl)-N-phenyl-2-sulfanylideneacetamide (4 g) and hydroxylamine hydrochloride (1.5 g) in ethanol (50 ml) at 80° C. The mixture was subsequently stirred at room temperature overnight before adding ethanedibromide (1.75 g) and triethylamine (6 ml). After heated under reflux for 6 h, the reaction mixture was cooled and concentrated to dryness to give an oil that gave a solid upon addition of diethyl ether. The solid was subsequently filtered, washed with more diethyl ether, and dried (5.8 g, mp 109-110° C.).

### Step 3: Preparation of N-(2,4-dichlorophenyl)-4,4-dioxo-5,6-dihydro-1λ⁶,4λ⁶-2-oxathiazine-3-carboxamide

A solution of N-phenyl-5,6-dihydro-1,4,2-oxathiazine-3-carboxamide (2.9 g) in dichloromethane (300 ml) was added gradually to a stirred solution of m-chloroperbenzoic acid (2.2 g) in dichloromethane (50 ml) at 0° C. The resulting mixture was then stirred at room temperature overnight before the excess peracid was destroyed with saturated aqueous NaHSO₃ (50 ml). The organic layer was separated, washed with saturated NaHCO₃ and water, dried (MgSO₄), and concentrated in vacuo to afford a colorless solid that was further washed with diethyl ether (2 g, mp 150-154° C.). ¹H-NMR (DMSO-d₆) δ 3.3 (m, 2H) 4.1 (dt, 1H), 4.8 (dt, 1H), 7.4 (m, 3H), 7.8 (m, 2H), 11.0 (br s, NH).

### Example 2

### Preparation of 4,4-dioxo-N-phenyl-5,6-dihydro-1, 4λ⁶,2-oxathiazine-3-carboxamide (Compound #4)

N-phenyl-5,6-dihydro-1,4,2-oxathiazine-3-carboxamide (2.9 g) isolated from Step 2 of Example 6 was oxidized as described in Step 3 of Example 1 except that two equivalents of m-chloroperbenzoic acid was used to give the desired product as a solid (2.7 g, mp 206-207° C.). ¹H-NMR (DMSO-d₆) δ 4.1 (m, 2H), 5.2 (m, 2H), 7.4 (m, 3H), 7.8 (m, 2H), 11.5 (br s, NH).

### Example 3

### Preparation of 3-[5-(3-chlorophenyl)-1,3,4-oxadiazol-2-yl]-5,6-dihydro-1,4λ⁴,2-oxathiazin-4-one (Compound #8)

### Step 1: Preparation of N′-[(3-chlorophenyl)carbonyl]-5,6-dihydro-1,4,2-oxathiazine-3-carbohydrazide

3-Chloro-N′-(chloroacetyl)benzohydrazide (15.6 g) was converted to N′-[(3-chlorophenyl)carbonyl]-5,6-dihydro-1,4,2-oxathiazine-3-carbohydrazide as a solid (10 g, mp 205-208° C.) using the procedures described in Steps 1 and 2 of Example 1.

### Step 2: Preparation of 3-[5-(3-chlorophenyl)-1,3,4-oxadiazol-2-yl]-5,6-dihydro-1,4,2-oxathiazine

N′-[(3-chlorophenyl)carbonyl]-5,6-dihydro-1,4,2-oxathiazine-3-carbohydrazide (10 g) was dissolved in POCl₃ (30 ml) and stirred at room temperature for 2 h, and further heated at reflux for 3 h. Excess POCl3 was brought to a lower volume under reduced pressure before the mixture was poured on ice and extracted with dichloromethane. The dried extract (MgSO₄) was concentrated to dryness to give a yellow solid which was further washed with a cold mixture of dichloromethane and diethyl ether (1:1) (4.5 g, mp 170-172° C.).

### Step 3: Preparation of 3-[5-(3-chlorophenyl)-1,3,4-oxadiazol-2-yl]-5,6-dihydro-1,4λ⁴,2-oxathiazin-4-one (Compound #8)

3-[5-(3-Chlorophenyl)-1,3,4-oxadiazol-2-yl]-5,6-dihydro-1,4,2-oxathiazine (2 g) was oxidized as described in Step 3 of Example 1 with one equivalent of m-chloroperbenzoic acid to give the desired product as a solid (1.8 g, mp 208-210° C.). ¹H-NMR (DMSO-d₆) δ 3.5 (m, 2H), 4.25 (dt, 1H), (dt, 1H), 7.75 (m, 2H), 8.1 (m, 2H).

### Example 4

### Preparation of 3-(3-bromo-1-benzothiophen-2-yl)-5,6-dihydro-1,4λ⁴,2-oxathiazin-4-one (Compound #13)

### Step 1: Preparation of 2-(methylsulfanyl)carbothioyl-1-benzothiophene

To a stirred solution of 1-benzothiophene (13.5 g) in anhydrous diethyl ether (120 ml) at room temperature was added 2.5 M nBuLi in hexanes (40 ml) dropwise over a period of 0.5 h. The mixture was stirred for 4 h and then cooled to −35° C. before adding dropwise a solution of carbon disulfide (6.2 ml) in diethyl ether (10 ml). The reaction solution was gradually warmed to room temperature stepwise over a period of 3 h. Methyl iodide (6.5 ml) in diethyl ether (10 ml) was then added dropwise and subsequently stirred at room temperature overnight. Water was then added and organic layer was separated, dried and concentrated to afford a red solid (20 g).

### Step 2: Preparation of 3-(1-benzothiophen-2-yl)-5,6-dihydro-1,4,2-oxathiazine

A solution of triethylamine (23 ml) in methanol (27 ml) was added dropwise to a stirred suspension of hydroxylamine hydrochloride (8 g) and 2-(methylsulfanyl)carbothioyl-1-benzothiophene (20 g) in methanol (180 ml) at room temperature. After 0.5 h, dibromoethane (7.7 ml) was added dropwise and the reaction mixture was stirred for a further 20 h before solvent was removed under reduced pressure. Water (35 ml) was added to the resulting residue and a brown solid formed was then filtered, dried, and washed with hot isopropanol to give a colorless solid (14 g).

### Step 3: Preparation of 3-(3-bromo-1-benzothiophen-2-yl)-5,6-dihydro-1,4,2-oxathiazine

A solution of bromine (4 ml) in chloroform (10 ml) was added dropwise to a stirred solution of 3-(1-benzothiophen-2-yl)-5,6-dihydro-1,4,2-oxathiazine (9.4 g) in chloroform (40 ml) over a period of 20 min. The mixture was stirred at room temperature overnight before it was basified with 2N NaOH and extracted with diethyl ether. The extract was washed with 1M sodium sulfite, water, dried (MgSO₄) and concentrated to afford a beige solid (12 g, mp 80-85° C.).

### Step 4: Preparation of 3-(3-bromo-1-benzothiophen-2-yl)-5,6-dihydro-1,4λ⁴,2-oxathiazin-4-one (Compound #13)

NaOCl (14%, 41 ml) was added dropwise to a stirred suspension of 3-(3-Bromo-1-benzothiophen-2-yl)-5,6-dihydro-1,4,2-oxathiazine (2 g) in ethyl acetate (20 ml) at 30° C. over a period of 20 min. The mixture was then stirred at room temperature overnight, concentrated to remove ethyl acetate, and then extracted with dichloromethane. The extract was dried (MgSO₄), concentrated to a solid which was further purified by column chromatography on silica gel (80% dichloromethane/diethyl ether) (1.9 g). ¹H-NMR (DMSO-d₆) δ 3.57 (m, 2H), 4.19 (dt, 1H), 4.77 (dt, 1H), 7.60 (m, 2H), 7.88 (dd, 1H), 8.13 (dd, 1H).

Ethyl 5,6-dihydro-3-phenyl-1,4-dithiin-2-carboxlate, 1,1,4,4-tetraoxide

### Example 5

### Preparation of ethyl 1,1,4,4-tetraoxo-3-phenyl-5,6-dihydro-1λ⁶,4λ⁶-dithiine-2carboxylate (Compound #17)

A mixture of ethanedithiol (2.5 g), ethyl 2-chloro-3-oxo-3-phenylpropanoate (5.6 g) and p-toluenesulphonic acid (0.1 g) in toluene (25 ml) was heated under reflux with a Dean-Stark trap for 4 h. During which time, water was constantly removed azeotropically. The mixture was then cooled to room temperature, washed with sodium bicarbonate saturated solution, dried (MgSO₄), and concentrated. The product was further purified by distillation to give a colorless oil (172-175° C./0.1 mm) which was then added to a solution mixture of 30% H₂O₂ (20 ml), glacial acetic acid (20 ml) and ethanol (20 ml). The mixture was heated on a steam bath for 0.5 h, and then stirred at room temperature overnight. The crystals formed were filtered and recrystallized from ethanol (5 g, mp 190-191° C.). ¹H-NMR (DMSO-d₆) δ 0.82 (t, 3H), 4.0 (q, 2H), 4.47 (m, 4H), 7.5 (m, 5H).

### Example 6

### Preparation of 2,3-diethyl 1,1,4,4-tetraoxo-5,6-dihydro-1λ⁶,4λ⁶-dithiine-2,3-dicarboxylate (Compound #20)

### Step 1: Preparation of 5,6-dihydro-1,4-dithiine-2,3-dicarbonitrile

To sodium cyanide (9.8 g) and carbon disulfide (15.2 g) in DMF (60 ml) was added n-butanol (100 ml). The mixture was stirred at room temperature overnight. The resulting solid was filtered, redissolved in water (100 ml), and the aqueous solution was allowed to sit at room temperature overnight and then filtered. Dioxane (0.15 g) was added to the resulting aqueous filtrate as wetting agent, and then followed by dropwise addition of 1,2-dibromoethane (17 g). The reaction temperature was maintained at 25° C. during the addition. The mixture was stirred overnight and filtered to give a brown solid after being washed with water and dried under vacuum (9.2 g, 50% yield).

### Step 2: Preparation of 2,3-diethyl 5,6-dihydro-1,4-dithiine-2,3-dicarboxylate

To a stirred solution of 5,6-dihydro-1,4-dithiine-2,3-dicarbonitrile (9.2 g) in concentrated sulfuric acid (100 ml) was added water (53 ml) gradually so that temperature did not exceed 110° C. The solution was cooled, poured on ice, and left standing at ice temperature overnight. The precipitate was filtered and redissolved in water (22 ml). Sodium hydroxide (5.3 g) was added to the solution and the resulting mixture was heated at reflux for 3 h, cooled, acidified with concentrated HCl to pH 1, and allowed to stand overnight. The resulting yellow precipitate was filtered, recrystallized from isopropanol, and redissolved in absolute ethanol (34 ml). To this ethanolic solution was added dropwise thionyl chloride (4.3 g) and then gradually heated to 78° C. overnight. The mixture was concentrated, ice water was added, and extracted with toluene. The toluene extract was dried over MgSO₄, concentrated, and distilled in vacuo to give a yellow oil (170° C./0.4 mm) that solidified on standing (2.7 g, mp 32-36° C.).

### Step 3: Preparation of 2,3-diethyl 1,1,4,4-tetraoxo-5,6-dihydro-1λ⁶,4λ⁶-dithiine-2,3-dicarboxylate (Compound 1)

2,3-Diethyl 5,6-dihydro-1,4-dithiine-2,3-dicarboxylate (2.7 g) was added slowly to a stirred solution of glacial acetic acid (9 ml) and 40% peracetic acid (9 ml) at room temperature. After 3 h stirring, water was added and the mixture was stored at 4° C. overnight. The colorless crystal formed was filtered and recrystallized from diethyl ether (0.3 g, mp 154-156° C.). ¹H-NMR (CDCl₃) δ 1.36 (t, 2×3H), 3.96 (s, 2×2H), 4.40 (q, 2×2H).

### Example 7

### Preparation of 2-methyl-3-(phenoxymethyl)-5,6-dihydro-1λ⁶,4λ⁶-dithiine-1,1,4,4-tetrone (Compound #21)

### Step 1: Preparation of ethyl 3-methyl-5,6-dihydro-1,4-dithiine-2-carboxylate

A mixture of ethanedithiol (3 g), ethyl 2-chloro-3-oxobutanoate (5 g), and p-toluenesulphonic acid (0.1 g) in toluene (30 ml) was heated under reflux with a Dean-Stark trap for 5 h. During which time, water was constantly removed azeotropically. The mixture was then cooled to room temperature, washed with sodium bicarbonate saturated solution, dried (MgSO₄), and concentrated. The product was further purified by distillation to give a colorless oil (6 g, 140-145° C./0.5 mm).

5,6-dihydro-2-methyl-3-(phenoxymethyl)-1,4-dithiin, 1,1,4,4-tetraoxide.

### Step 2: Preparation of (3-methyl-5,6-dihydro-1,4-dithiin-2-yl)methanol

A solution of ethyl 3-methyl-5,6-dihydro-1,4-dithiine-2-carboxylate (6 g) in diethyl ether (10 ml) was added dropwise to a stirred solution of LiAlH₄ (2 g) in diethyl ether (30 ml) at 10° C. The reaction was allowed to proceed at room temperature overnight, quenched with water and extracted with diethyl ether. After the solvent was removed, the liquid residue was purified by distillation to give an oil (2 g, by 125-128° C./0.05 mm).

### Step 3: Preparation of 2-methyl-3-(phenoxymethyl)-5,6-dihydro-1λ⁶,4λ⁶-dithiine-1,1,4,4-tetrone (Compound #2])

A mixture of (3-methyl-5,6-dihydro-1,4-dithiin-2-yl)methanol (2 g), 4-tert-butylphenol (1.6 g), DMAP (0.2 g), and DCC (2 g) in THF (50 ml) was heated under reflux overnight. After solvent was removed under reduced pressure, the residue was purified by column chromatography on silica gel to give a solid (0.8 g). The solid was then dissolved in acetic acid (7 ml)/40% peracetic acid (7 ml) and heated to 90° C. for 0.5 h. Water (30 ml) was added and the mixture was filtered to give a solid (0.8 g). ¹H-NMR (DMSO-d₆) δ 1.24 (s, 9H), 2.18 (s, 3H), 4.23 (m, 4H), 4.95 (s, 2H), 6.97 (d, 2H), 7.37 (d, 2H).

### Example 8

### Preparation of ethyl 1,1,4,4-tetraoxo-3-(trifluoromethyl)-5,6-dihydro-1λ⁶,4λ⁶-dithiine-2-carboxylate (Compound #24)

A mixture of ethanedithiol (2.2 g), ethyl 2-chloro-4,4,4-trifluoro-3-oxobutanoate (5 g), and p-toluenesulphonic acid (0.1 g) in toluene (30 ml) was heated under reflux with a Dean-Stark trap for 2 days. During which time, water was constantly removed azeotropically. The mixture was then cooled to room temperature, washed with sodium bicarbonate saturated solution, dried (MgSO₄), and concentrated. The product was further purified by distillation to give a colorless oil (172-175° C./0.1 mm) which was then added to a solution mixture of 30% H₂O₂ (20 ml), glacial acetic acid (20 ml) and ethanol (20 ml). The oxidation reaction was carried out a steam bath for 1 h, then at room temperature overnight. The crystals formed were filtered and recrystallized from ethanol (4.5 g, mp 142-145° C.). ¹H-NMR (DMSO-d₆) δ 1.29 (t, 3H), 4.45 (q, 2H), 4.53 (m, 4H).

### Example 9

### Preparation of 1,1,4,4-tetraoxo-N-phenyl-3-(trifluoromethyl)-5,6-dihydro-1λ⁶,4λ⁶-dithiine-2-carboxamide (Compound # 25)

A mixture of ethyl 1,1,4,4-tetraoxo-3-(trifluoromethyl)-5,6-dihydro-1λ⁶,4λ⁶-dithiine-2-carboxylate (Compound 3) (1 g), potassium hydroxide (2.5 g), ethanol (5 ml) and water (5 ml) was stirred at room temperature for two days. The mixture was concentrated in vacuo to remove ethanol, and then acidified with 5% HCl (aq) at 0° C. to pH 1. The resulting solid was filtered, washed with water, and dried under vacuum (0.8 g, mp 105-108° C.). The dried solid was redissolved in thionyl chloride (30 ml) with a trace amount of pyridine (1 drop) added, stirred at room temperature overnight, and then concentrated to dryness. The residue was redissolved in dichloromethane (5 ml) and added dropwise to a stirred solution of aniline (0.33 g) and pyridine (0.5 ml) in dichloromethane (10 ml) at room temperature. After stifling overnight, the reaction mixture was washed with water (3×10 ml), dried (MgSO₄) and concentrated. The resulting solid was purified by recrystallization from ethanol (0.8 g, mp 169-171), and then oxidized in a solution mixture of 30% H₂O₂ (20 ml), glacial acetic acid (20 ml) and ethanol (20 ml) on steam bath for 12 h. The reaction mixture was concentrated to dryness and the resulting solid was recrystallized from acetonitrile and water (0.7 g, mp 217-219° C.). ¹H-NMR (DMSO-d₆) δ 4.4 (m, 4H), 7.4 (m, 5H), 11.3 (br s, NH).

### Examples of Biological Testing

### Example 10

### Fluorescent Spectroscopy Studies on the Covalent Labeling of Sulfhydryl Groups of Biomolecules

The ability of drugs to label sulfhydryl groups of cysteine, glutathione, human serum albumin (HSA) and biomolecules in K562 cells was determined spectrofluorometrically using Thioglo-1, a maleimide reagent that reacts quickly with the sulfhydryl groups to produce highly fluorescent covalent adducts. Drugs that react with sulfhydryl groups block the formation of fluorescent adducts when sulfhydryl reactant is treated with drugs prior to Thioglo-1 treatment. Cysteine (10 μM), glutathione (10 μM), human serum albumin (10 μM), or K562 cell homogenate (6×10⁵ cells) in 20 mM Tris pH 8.0, was treated with 1μl of drug (or not) in DMSO at 50 μM reaction concentration at 37° C. for 3 h, followed by the addition of Thioglo-1 (22 μM). The fluorescence was measured in a Fluostar Galaxy (BMG, Durham, N.C.) fluorescence plate reader using an excitation wavelength of 380 nm and an emission wavelength of 520 nm, and the percentage inhibition of fluorescent labeling was obtained using the following equation: % Inhibition=[(1−(F_{drug}−F_{background})/F_{dmso}]×100, where F_{drug} is the fluorescent value for Thioglo-1 treatment of biomolecules pretreated with drug, F_{background} is the fluorescent value for sample containing Thioglo-1 in Tris buffer only, and F_{dmso} is the fluorescent value for Thioglo-1 treatment of biomolecules pretreated with DMSO solvent only.

### Example 11

### Electrospray Ionization Mass Spectrometry (ESI-MS) Studies on the Covalent Labeling of Sulfhydryl Group of Protein

The MS drug-HSA protein binding studies were carried out using an Applied Biosystems API 2000 Triple Quadrupole mass spectrometer (Thornhill, Canada) equipped with a syringe pump (Harvade Apparatus, Holliston, Mass.) at a flow rate of 5-10 μl/min. The Analyst software (version 1.4) was used for system control and data acquisition. The ESI source was operated in the positive ion mode with an electrospray voltage of +4.4 kV without capillary heating. MagTran freeware (version 1.02, http://www.geocities.com/SiliconValley/Hills/2679/magtran.html) was used for charge state deconvolution of HSA and drug-HSA covalent adducts.

The reaction of drug and HSA was carried out by mixing drug (or not) in DMSO (11μl, 6 mM) with HSA in 16 mM Tris pH 7.5 buffer (1 ml, 65 μM) for 5 h at room temperature. The reaction mixture was then dialyzed using a dialysis membrane with a 10,000 Da cutoff. The dialyzed mixture (150 μl) was diluted with a solution mixture of water (124 μl), methanol (76 μl), and formic acid (17 μl, 6% v/v). Scanning was 1000-1800 m/z units every 4 s with a step size of 0.10 amu.

### Example 12

### Topoisomerase IIα Decatenation Inhibition Assay

The catalytic inhibition of human topoisomerase IIα by a drug was measured by the ATP-dependent decatenation of kDNA (Topogen, Columbus, Ohio) into minicircles of DNA as we previously described (Hasinoff 1995 QSAR ICRF-187). The 20 μl reaction mixture contained 0.5 mM ATP, 50 mM Tris-HCl (pH 8.0), 120 mM KCl, 10 mM MgCl₂, 30 μg/ml bovine serum albumin, 40 ng kDNA, drug or DMSO (0.5 μl) and 300 ng K562 cells nuclear extract, the amount that gave 80% decatenation. The enzymatic reaction was carried out at 37° C. and was terminated by the addition of 6 μl of buffer containing 5 mM Tris pH 8.0, 30% w/v sucrose, 0.5% bromophenol blue, and 125 mM EDTA. The resulting mixture was separated by electrophoresis (2 h at 8 V/cm) on an agarose gel prepared from 1.2% w/v agarose and 0.5 μg/ml ethidium bromide in TAE buffer pH 8.0 (40 mM Tris base, 0.114% (v/v) glacial acetic acid, 2 mM EDTA). The DNA in the gel was imaged by its fluorescence on an Alpha Annotech Fluorchem 8900 imaging system equipped with a 365 nm illuminator and a CCD camera. Densitometry scanning of gel photographs was used to obtain the fluorescence intensity of the band corresponding to the DNA minicircles. The percentage inhibition of K562 topoisomerase II catalytic activity at concentrations of 3 and 30 μM was determined using the following equation: % Inhibition=[(1−(B_{drug}−B_{background})/B_{dmso}]×100, where B_{drug} is the band intensity value for the enzymatic reaction sample treated with drug, B_{background} is the band intensity value for sample without the enzyme, and B_{dmso} is the band intensity value for the enzymatic reaction sample treated with DMSO solvent only.

### Example 13

### Cell Culture and Growth Inhibition Assay

K562 cells were obtained from American Type Culture Collection (Rockville, Md.). These cells were maintained as suspension cultures in alpha minimum essential medium (αMEM) (Gibco BRL, Burlington, Canada) containing 2 mM L-glutamine and supplemented with 10% fetal calf serum (Invitrogen, Burlington, ON, Canada), 20 mM NaHCO₃, 20 mM HEPES (Sigma), 100 units/ml penicillin G, and 100 μg/ml streptomycin at pH 7.4 in an atmosphere of 5% CO₂ and 95% air at 37° C. For the measurement of growth inhibition, cells in exponential growth were harvested and seeded at 6000 cells/well in 96-well plates (100 μl/well). Drugs were dissolved in DMSO and added to the wells such that the final concentration of DMSO was 0.5% (v/v). After 72 h incubation, 7 μl of 3-(4,5-dimethylthiazol-2-yl)-5-(3-carboxymethoxyphenyl)-2-(4-sulfophenyl)-2H-tetrazolium (MTS) Cell Titer 96® AQueous One Solution (Promega, Md., WI) was added to each well and incubated for a further 3 h. The absorbance was measured in a Molecular Devices (Menlo Park, Calif.) plate reader. The spectrophotometric 96-well plate cell growth inhibition assay measures the ability of the cells to enzymatically reduce MTS. Three replicates were measured at each drug concentration, and the IC₅₀ values and their SEs for growth inhibition were obtained by fitting the absorbance-drug concentration data to a four-parameter logistic equation.

TABLE 1
Illustrative compounds of the invention

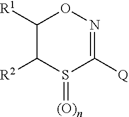

| CMPD # | Q | n | R1 | R2 | mp (° C.) |
|---|---|---|---|---|---|
| 1 | C₆H₅ | 1 | H | H | 68-70 |
| 2 | 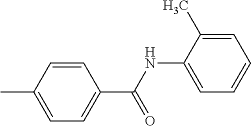 | 2 | H | H | 212-213 |
| 3 | CONHC₆H₅ | 1 | H | H | 150-154 |
| 4 | CONHC₆H₅ | 2 | H | H | 206-207 |
| 5 | 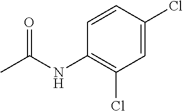 | 2 | H | H | 199-200 |
| 6 | 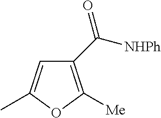 | 1 | H | H | 182-183 |
| 7 | 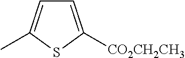 | 1 | H | H | 109-110 |
| 8 | 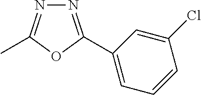 | 1 | H | H | 208-210 |

TABLE 2
Illustrative compounds of the invention

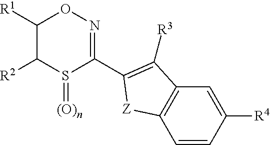

| CMPD # | n | R¹ | R² | R³ | R² | Z | mp (° C.) | NMR (DMSO-d6) |
|---|---|---|---|---|---|---|---|---|
| 9 | 1 | H | H | H | H | S | 140-142 | |
| 10 | 2 | H | H | H | H | S | 150-154 | |
| 11 | 1 | CH₃ | H | H | H | S | 150-153 | |
| 12 | 1 | H | H | CH₃ | CH₃ | S | 133-135 | |
| 13 | 1 | H | H | Br | H | S | | 3.57 (m, 2H), 4.19 (dt, 1H), |
| | | | | | | | | 4.77 (dt, 1H), 7.60 (m, 2H), |
| | | | | | | | | 7.88 (dd, 1H), 8.13 (dd, 1H) |
| 14 | 1 | H | H | H | H | SO | | 3.54 (m, 2H), 4.15 (dt,1H), |
| | | | | | | | | 4.75 (dt, 1H), 7.65 (m, 2H), |
| | | | | | | | | 7.86 (dd, 1H), 8.06 (dd, 1H), 8.13 (s, 1H) |
| 15 | 2 | H | H | H | H | SO₂ | | 4.34 (m, 2H), 5.06 (m, 2H), |
| | | | | | | | | 7.77 (m, 2H), 7.95 (m, 2H), |
| | | | | | | | | 8.26 (s, 1H) |

TABLE 3
Illustrative compounds of the invention

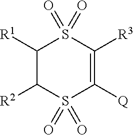

| | | | | | | NMR |
|---|---|---|---|---|---|---|
| CMPD # | Q | R¹ | R² | R³ | mp (° C.) | (DMSO-d₆) |
| 16 | C₆H₅ | C₂H₅ | H | C₆H₅ | 145-148 | |
| 17 | C₆H₅ | H | H | CO₂CH₂CH₃ | 190-191 | |
| 18 | C₆H₅ | H | H | nC₄H₉ | 139-141 | |
| 19 | C₆H₅ | CH₃ | CH₃ | H | 106-112 | |
| 20 | CO₂CH₂CH₃ | H | H | CO₂CH₂CH₃ | 154-156 | |
| 21 | 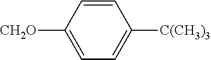 | H | H | CH₃ | | 1.24 (s, 9H), 2.18 (s, 3H), 4.23 (m, 4H), 4.95 (s, 2H), 6.97 (d, 2H), 7.37 (d, 2H) |
| 22 | CH₂SO₂C₆H₅ | H | H | CH₃ | 226-228 | |
| 23 | CH₂OCH₃ | H | H | CH₂OCH₃ | | 3.29 (s, 6H), |
| | | | | | | 4.18 (s, 4H), |
| | | | | | | 4.39 (s, 4H) |
| 24 | CO₂CH₂CH₃ | H | H | CF₃ | 142-145 | |
| 25 | CONHC₆H₅ | H | H | CF₃ | 217-219 | |

TABLE 4
Drug inhibition of fluorescent labeling of biomolecules
containing sulfhydryl groups

| | % Inhibition of Thioglo-1 fluorescent labeling of | | | |
|---|---|---|---|---|
| | | | Human | |
| | 10 μM | 10 μM | serum | K562 Cell |
| Drug | Cysteine | Glutathione | albumin | homogenate |
| 50 μM CMPD #9 | 100 | 100 | 85 | 95 |
| DMSO control | 0 | 0 | 0 | 0 |

TABLE 5
Electrospray Ionization Mass Spectrometry (ESI-MS) studies on the covalent labeling
of the sulfhydryl group of human serum albumin (HSA). Table shows the experimental and
calculated molecular weights of peaks observed in the deconvoluted molecular ion regions of
HSA treated with DMSO and Compound 9, as shown in spectra B and D, respectively. A and C
are ESI positive-ion mass spectra of HSA and HSA treated with compound 9, respectively.

| | | MW (Da) | MW (Da) |
|---|---|---|---|
| Peak | Description | experimental | calculated |
| HSA | Free HSA | 66,398 | 66,430 |
| HSA − 2H + Cys | Cysteinylated HSA, a post-translational | 66,542 | 66,549 |
| | modification product present in the HSA | | |
| HAS + Compound 9 | Covalent adduct of HSA and Compound 9 | 66,672 | 66,681 |

TABLE 6
Drug inhibition of human topoisomerase II catalysis and
human leukemia K562 cell growth.

| | % Inhibition of topoisomerase | | Median growth |
|---|---|---|---|
| CMPD | II catalytic activity | | inhibitory concentration |
| # | 30 μM | 3 μM | (IC₅₀) of K562 cells (μM) |
| 1 | 100 | 70 | 11 |
| 2 | 100 | 100 | 1 |
| 3 | 100 | 100 | 1 |
| 4 | 100 | 100 | 0.7 |
| 5 | 100 | 100 | 1 |
| 6 | 100 | 100 | 2 |
| 7 | 100 | 100 | 0.9 |
| 8 | 100 | 80 | 4 |
| 9 | 100 | 100 | 0.7 |
| 10 | 100 | 100 | 0.5 |
| 11 | 100 | 100 | 2 |
| 12 | 100 | 100 | 0.7 |
| 13 | 100 | 100 | 1 |
| 14 | 100 | 100 | 1 |
| 15 | 100 | 100 | 0.5 |
| 16 | 100 | 80 | 4 |
| 17 | 100 | 100 | 3 |
| 18 | 100 | 70 | 5 |
| 19 | 100 | 100 | 4 |
| 20 | 100 | 100 | 1 |
| 21 | 100 | 100 | 0.6 |
| 22 | 100 | 100 | 1 |
| 23 | 100 | 100 | 2.3 |
| 24 | 100 | 100 | 2.5 |
| 25 | 100 | 100 | 4.0 |

What is claimed is:

1. A compound of the formula: 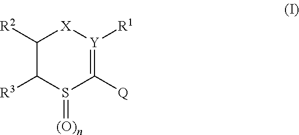
wherein
X is oxygen or sulfone;
Y is nitrogen when X is oxygen, or carbon when X is sulfone;
n is 1 or 2, with the proviso that when n is 1, X must be oxygen and Y must be nitrogen;
R¹ is present when Y is carbon, or absent when Y is nitrogen, and when present is hydrogen, C₁-C₆ linear or branched alkyl, phenyl, trihalomethyl, cyano, benzyl, phenylsulfone, methyl sulfone, methyl alcohol, nitro, methylene C₁-C₆ alkoxy, methylene C₁-C₆ thioalkoxy, methylene benzyloxy, methylene phenoxy, or methylene acetate; R² and R³ are each independently hydrogen, C₁-C₆ linear or branched alkyl, benzyl, methylene C₁-C₄ alkoxy, or halogen;
Q is
(a) phenyl, with the proviso that R¹ is present and cannot be hydrogen, straight chain or branched alkyl, or substituted or unsubstituted phenyl; 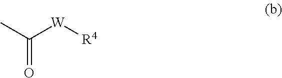
wherein W is oxygen, amino, C₁-0₅ linear or branched alkylamino, or CH₂; R⁴ is hydrogen, C₁-C₆ linear or branched alkyl, C₅-C₆ cycloalkyl, ethylphenyl, or phenyl optionally substituted with 1 to 3 substituents independently selected from halogen, C₁-C₆ alkyl, C₁-C₆ alkoxyl, C₁-C₆ linear or branched alkoxylcarbonyl, trihalomethyl, phenyl, nitro, or aminoacetyl, with the proviso that R¹ may be present and if present cannot be hydrogen, straight chain or branched alkyl, cyano, or substituted or unsubstituted phenyl;
(c) 1,3,4-oxadiazole substituted with phenyl or halophenyl; 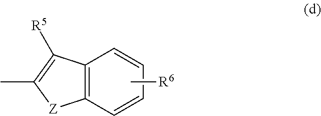
wherein Z is sulfur, sulfoxide, or sulfone; R⁵ is hydrogen, C₁-C₄ linear alkyl, C₁-C₄ branched alkoxy, halogen, haloalkyl, nitro, phenyl, or methylene halophenoxy; R⁶ is hydrogen, C₁-C₄ alkyl, halogen, or trihalomethyl, with the proviso that when Z is sulfur, R⁵ cannot be hydrogen or C₁-C₄ branched alkoxy, and R⁶ cannot be hydrogen or C₁-C₄ alkyl; 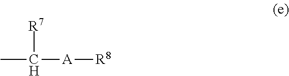
wherein A is oxygen or sulfone; R⁷ is hydrogen or C₁-C₄ alkoxy; R⁸ is C₁-C₆ linear or branched alkyl, phenyl, biphenyl, halophenyl, C₁-C₄ alkylphenyl, benzyl, C₁-C₄ alkylcarbonyl, phenylcarbonyl, C₁-C₄ alkylaminocarbonyl, or phenylaminocarbonyl.

2. A method of treating a human disease disorder mediated by a sulfhydryl-dependent biomolecule in a subject in need thereof comprising administering to the subject an effective amount of a compound of structural formula: 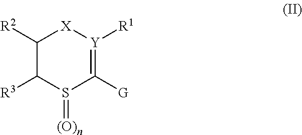
wherein
X is oxygen or sulfone;
Y is nitrogen when X is oxygen, or carbon when X is sulfone;
n is 1 or 2, with the proviso that when n is 1, X must be oxygen and Y must be nitrogen;
R¹ is present when Y is carbon, or absent when Y is nitrogen, and when present is a hydrogen, C₁-C₆ linear or branched alkyl, C₁-C₃ haloalkyl, trihalomethyl, benzyl, phenylsulfone, methyl sulfone, methyl alcohol, nitro, methylene C₁-C₆ alkoxy, methylene C₁-C₆ thioalkoxy, methylene benzyloxy, methylene phenoxy, methylene acetate, C₁-C₆ alkoxycarbonyl, phenyl, nitrophenyl, halophenyl, C₁-C₄ alkylphenyl, C₁-C₄ alkoxyphenyl, or naphthyl; R² and R³ are each independently hydrogen, C₁-C₆ linear or branched alkyl, benzyl, methylene C₁-C₄ alkoxy, or halogen;
G is
(a) phenyl, naphthyl or pyridinyl; phenyl optionally substituted with 1 to 3 substituents independently selected from halogen, C₁-C₁₂ linear or branched alkyl, C₅-C₆ cycloalkyl, haloalkyl, phenyl, C₁-C₄ alkoxy, C₁-C₄ thioalkoxy, tetrahydrophyranyloxy, phenoxy, C₁-C₅ alkylcarbonyl, C₁-C₅ alkoxycarbonyl; C₁-C₅ alkylaminocarbonyl, phenylaminocarbonyl, tolylamonicarbonyl, morpholinocarbonyl, amino, nitro, cyano, dioxolanyl; 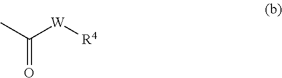
wherein W is oxygen, amino, C₁-C₅ linear or branched alkylamino, or CH₂; R⁴ is hydrogen, C₁-C₆ linear or branched alkyl, C₅-C₆ cycloalkyl, ethylphenyl, or phenyl optionally substituted with 1 to 3 substituents independently selected from halogen, C₁-C₆ alkyl, C₁-C₆ alkoxyl, C₁-C₆ linear or branched alkoxylcarbonyl, trihalomethyl, phenyl, nitro, or aminoacetyl, with the proviso that R¹ cannot be hydrogen, straight chain or branched alkyl, or cyano;
(c) thienyl or furanyl, each optionally substituted with 1 to 3 substituents independently selected from halogen, C₁-C₆ linear or branched alkyl, C₁-C₅ alkoxy, C₁-C₅ thioalkoxy, trihalomethyl, C₁-C₆ alkoxycarbonyl, cyano, acetyl, formyl, benzoyl, nitro, phenylaminocarbonyl, or phenyl;
(d) 1,3,4-oxadiazole substituted with phenyl or halophenyl; 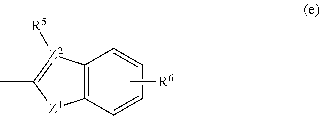
wherein Z¹ is oxygen, sulfur, sulfoxide, or sulfone; Z² is nitrogen or carbon; R⁵ is present when Z² is carbon, or absent when Z² is nitrogen, and when present is a hydrogen, C₁-C₄ linear alkyl, C₁-C₄ branched alkoxy, halogen, haloalkyl, nitro, phenyl, or methylene halophenoxy; R⁶ is hydrogen, C₁-C₄ alkyl, halogen, or trihalomethyl; 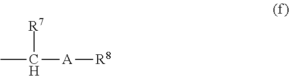
wherein A is oxygen or sulfone; R⁷ is hydrogen or C₁-C₄ alkoxy; R⁸ is C₁-C₆ linear or branched alkyl, phenyl, biphenyl, halophenyl, C₁-C₄ alkylphenyl, benzyl, C₁-C₄ alkylcarbonyl, phenylcarbonyl, C₁-C₄ alkylaminocarbonyl, or phenylaminocarbonyl.

3. A method according to claim 2 in which the human disease disorder is cancer.
